(19) United States
(12) Patent Application Publication
(10) Pub. No.: US 2022/0127293 A1
(43) Pub. Date: Apr. 28, 2022

(54) LIQUID METAL COMPLEX HAVING OXYGEN-ABSORBING ABILITY

(71) Applicants: SHARP KABUSHIKI KAISHA, Sakai City, Osaka (JP); NATIONAL UNIVERSITY CORPORATION KOBE UNIVERSITY, Kobe-shi, Hyogo (JP)

(72) Inventors: Yasunori NAKANISHI, Sakai City, Osaka (JP); Hideto MATSUYAMA, Kobe-shi, Hyogo (JP); Eiji KAMIO, Kobe-shi, Hyogo (JP); Atsushi MATSUOKA, Kobe-shi, Hyogo (JP)

(21) Appl. No.: 17/438,809

(22) PCT Filed: Mar. 4, 2020
(86) PCT No.: PCT/JP2020/009190

§ 371 Date: Sep. 13, 2021

(30) Foreign Application Priority Data
Mar. 14, 2019 (JP) 2019-047618

(51) Int. Cl. C07F 15/06; B01D 53/14

(52) U.S. Cl. CPC C07F 15/065; B01D 2253/20; B01D 2257/104; B01D 53/1493

(57) ABSTRACT

To provide a liquid metal complex having an oxygen absorbing ability, containing a cobalt-acacen complex or a derivative thereof, and an ionic liquid in which an ionic ligand having an amine structure and a counter ion thereof are paired, in which the cobalt-acacen complex or the derivative thereof is expressed by general formula (1):

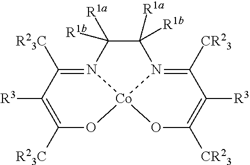

and the liquid metal complex has a structure in which the amine structure of the ionic ligand is axially coordinated with a cobalt atom of the cobalt-acacen complex or the derivative thereof.

### TECHNICAL FIELD

The present invention relates to a liquid metal complex having a molecular structure with low viscosity and an excellent oxygen-absorbing ability (hereinafter, also referred to as a “oxygen absorbing ability”).

### BACKGROUND ART

A technology for separating and recovering a mixed gas and an application process of the technology can be used for multiple purposes such as reduced pollutant and greenhouse gas emissions, deodorizing treatment, extraction of specific components, reaction promotion, and energy saving, and thus, its utilization can be found in a wide range of fields such as industrial plants, automobiles, chemistry, biotechnology, food, and medicine.

The most familiar mixed gas in our lives is air including nitrogen and oxygen. A method for separating the air into nitrogen and oxygen includes a cryogenic separation method suitable for mass production on a large scale, and an adsorption separation method and a membrane separation method on a small scale, and these methods are used properly according to each feature. In particular, the membrane separation method enables an advanced energy-saving and low-cost process compared to the other methods and enables a simple and compact device configuration, and thus, the method is expected to be applied to applications requiring weight reduction and miniaturization.

The separation performance in the membrane separation method depends on the design of a device system and a separation module, and the performance of a membrane material is the most important. Many membrane materials, in many cases, polymers, which can be used as oxygen separation membranes, have been developed and are available on the market, but with their current performance, these materials are still used only in limited applications and places.

Generally, a fact that the separation performance of a mixed gas in a polymer material is based on a trade-off between an amount of permeation and a separation selectivity ratio is summarized based on experimental data of previous studies (see NPL 1). There is not much difference in molecular size and molecular weight between nitrogen and oxygen, and thus, it is not easy to increase the separation selectivity ratio. Therefore, some kind of molecular action for selectively promoting permeation of oxygen is required to realize the performance exceeding the upper limit of such a performance.

A material having excellent oxygen absorption and being capable of reversibly and selectively absorbing and desorbing oxygen includes metal complex materials. For example, a cobalt-salen complex containing cobalt and a salen ligand is a material having high adsorption performance to oxygen, and an oxygen adsorbent and an oxygen separation method using the cobalt-salen complex have been proposed (see PTL 1 and PTL 2). Therefore, to fully obtain the oxygen absorption performance of the cobalt-salen complex, the present inventors have realized the liquefaction of the complex molecule by a structure in which an ionic liquid having an amine structure is coordinated with cobalt ions (see PTL 3).

A complex in a liquid state has higher molecular mobility than a complex in a solid state or in an immobilized state, and has fluidity in a porous membrane when the complex is encapsulated in the porous membrane. Therefore, it is considered that oxygen in the air permeates faster than nitrogen according to the following mechanism. First, oxygen in the air is attracted to an oxygen adsorption site of the complex encapsulated in the porous membrane and absorbed by the porous membrane. Thereafter, the oxygen adsorbed on the complex moves through the complex molecules or moves in gaps between the complex molecules to efficiently move in the membrane. As a result, the oxygen flowing to the downstream side of the membrane permeates the membrane faster than nitrogen.

Thus, a highly fluid complex serves as a carrier carrying oxygen, and moreover, the complex itself has fluidity to function as carriers accumulated at high density in the membrane, and thus, it is possible to exhibit excellent oxygen permeability.

Further, as an oxygen selective adsorbent, a cobalt-acacen complex containing cobalt and an acacen ligand has been proposed (see PTL 4). However, its form is solid, and liquefaction is desired to improve an oxygen absorbing ability.

### CITATION LIST

### Patent Literature

### [PTL 1]

Japanese Unexamined Patent Application Publication No. H9-151192

### [PTL 2]

Japanese Unexamined Patent Application Publication No. H6-340683

### [PTL 3]

### International Publication No. 2017/130833

### [PTL 4]

Japanese Unexamined Patent Application Publication No. H8-206496

### Non-Patent Literature

### [NPL 1]

Lloyd M. Robeson “The upper bound revisited”, Journal of Membrane Science, 2008, Vol. 320, p. 390-400

### SUMMARY OF THE INVENTION

### Problems to be Solved by the Invention

As described above, the metal complex is generally in a solid state, and the liquefaction is a major issue in its application. That is, the solubility in a solvent to be applied and the stability thereto, and a functional structure in the solution are important for exhibiting the original functionality of the metal complex. In a gas separation membrane, it is desired that a high-concentration solution can be prepared to utilize the complex at a high density. However, many metal complexes having a large molecular structure are usually not very soluble. On the other hand, when the metal complex itself is liquefied by forming a composite structure in which an ionic liquid is coordinated to the metal complex as in PTL 3, it is possible to treat the metal complex as a liquid without need of diluting the metal complex with another solvent, and as a result, it is possible to obtain a liquid in which the complex structure is densely integrated. Further, the metal complex is liquefied using an ionic liquid, and thus, the metal complex lacks volatility and has high liquid stability, which is also a practical advantage.

However, a liquid containing a complex structure site containing cobalt, salen, and a salen derivative is a fairly bulky molecule as one functional structure molecule, and also has a strong intermolecular action, so that the liquid is classified as a liquid having a considerably high viscosity.

To further improve the membrane performance by using the liquid complex as the oxygen separation membrane, it is necessary to enhance the fluidity of the complex serving as an oxygen carrier and the diffusivity of oxygen. That is, it is necessary to reduce the viscosity of the liquid complex to improve the membrane performance, but in the structure containing the above-mentioned salen complex and its derivative, it is no possible to realistically expect much structural improvement to further reduce the viscosity, given the influence of the size of the basic functional structure.

Therefore, an object of the present invention is to provide a liquid metal complex having a molecular structure with low viscosity and an excellent oxygen absorbing ability.

### Solution to Problem

To further improve separation performance, the inventors of the present invention examined, in a functional structure having an oxygen absorbing ability and a liquid complex containing the same, a molecular structure with low viscosity, which was significant for improving oxygen diffusion, in a low-viscosity liquid structure composition required to obtain its oxygen absorbing ability, that is, a liquid metal complex having an oxygen absorbing ability, and as a result, found that a metal complex in which an ionic liquid containing an amine structure is coordinated with a cobalt complex having an acacen skeleton being a structure obtained through a dehydration condensation reaction between ethylenediamine and acetylacetone, was a low-viscosity liquid and yet had oxygen absorption, and thus, the present invention has been reached.

Thus, according to the present invention, it is possible to provide a liquid metal complex having an oxygen absorbing ability, the liquid metal complex containing a cobalt-acacen complex or a derivative thereof, and an ionic liquid in which an ionic ligand having an amine structure and a counter ion thereof are paired, in which the cobalt-acacen complex or the derivative thereof is expressed by general formula (1):

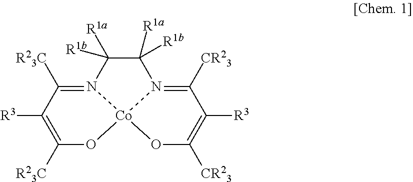

(where R^{1a}, R^{1b} and R³ are each independently a hydrogen atom, a halogen atom, an alkyl group or a haloalkyl group having 1 to 6 carbon atoms, an alkoxy group having 1 to 6 carbon atoms, an acyl group having 2 to 6 carbon atoms, or an alkoxycarbonyl group having 2 to 6 carbon atoms, R^{1a} and R^{1b} may be bonded to each other via an atom or an atomic group bonded to R^{1a} and R^{1b} to form a cycloalkyl ring, and R² is independently a hydrogen atom, a halogen atom, an alkyl group or a haloalkyl group having 1 to 6 carbon atoms, or an alkoxy group having 1 to 6 carbon atoms), and

the liquid metal complex has a structure in which the amine structure of the ionic ligand is axially coordinated with a cobalt atom of the cobalt-acacen complex or the derivative thereof.

### Effect of the Invention

According to the present invention, it is possible to provide a liquid metal complex having a molecular structure with low viscosity and an excellent oxygen absorbing ability.

That is, according to the present invention, it is possible to provide a low-viscosity liquid metal complex including a cobalt complex in a stable liquid state as a material selectively and reversibly absorbing oxygen, more specifically, including a cobalt-acacen complex or a derivative thereof and an ionic liquid having an amine structure, by which it is possible to efficiently absorb and diffuse oxygen.

Further, the liquid metal complex having an oxygen absorbing ability according to the present invention further exerts the above-mentioned effect when any one of the following requirements is satisfied.

(1) The ionic ligand is an ammonium cation having an alkyl group having 2 to 6 carbon atoms.
(2) The amine structure of the ionic ligand is a secondary amine.
(3) The ionic ligand is an N-methyl amino acid.
(4) The ionic ligand is a heterocyclic compound containing a structure of imidazole or pyridine.
(5) The counter ion contains an anion of bis(trifluoromethanesulfonyl)imide.
(6) The counter ion contains a phosphonium cation.
(7) The cobalt-acacen complex is a cobalt complex containing a cobalt atom and an acacen ligand obtained through a dehydration condensation reaction of ethylenediamine and acetylacetone.
(8) The liquid metal complex having an oxygen absorbing ability has a viscosity lower than 10000 mPa·s.

### BRIEF DESCRIPTION OF DRAWINGS

FIG. 1 is a schematic diagram of an absorption test device for measuring an oxygen absorption amount.

### MODE FOR CARRYING OUT THE INVENTION

Embodiments of the present invention will be described below in detail, but the present invention is not limited thereto.

(Liquid Metal Complex with Oxygen Absorbing Ability)

A liquid metal complex having an oxygen absorbing ability (also referred to as “oxygen absorbing liquid”) according to the present invention is characterized by including a cobalt-acacen complex or a derivative thereof (hereinafter, including the derivative, also referred to as “cobalt-acacen complex”), and an ionic liquid in which an ionic ligand having an amine structure and a counter ion thereof are paired, in which the liquid metal complex has a structure in which the amine structure of the ionic ligand is axially coordinated with a cobalt atom of the cobalt-acacen complex or the derivative thereof.

In the present invention, the “derivative of the cobalt-acacen complex” means a cobalt-acacen complex in which a substituent is introduced into an acacen skeleton obtained through a dehydration condensation reaction of acetylacetone and ethylenediamine.

When an ionic ligand having an amine structure is bonded in one axial direction of a cobalt atom of the cobalt-acacen complex, the opposite axial direction serves as an adsorption site for oxygen molecules, and when the adsorption site is reacted with oxygen molecules, a selective affinity with oxygen occurs. The basicity of the amine structure coordinated with the cobalt at this time affects the magnitude of the affinity action with oxygen.

### (Cobalt-Acacen Complex)

The cobalt-acacen complex that can be used in the present invention is a well-known metal complex having a structure in which an acacen derivative obtained by introducing a substituent into an acacen or an acacen skeleton structure is coordinated, as a tetradentate ligand, with a cobalt(II) ion, and is expressed by general formula (1):

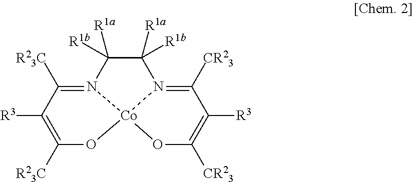

(where R^{1a}, R^{1b} and R³ are each independently a hydrogen atom, a halogen atom, an alkyl group or a haloalkyl group having 1 to 6 carbon atoms, an alkoxy group having 1 to 6 carbon atoms, an acyl group having 2 to 6 carbon atoms, or an alkoxycarbonyl group having 2 to 6 carbon atoms, R^{1a} and R^{1b} may be bonded to each other via an atom or an atomic group bonded to R^{1a} and R^{1b} to form a cycloalkyl ring, and R² is independently a hydrogen atom, a halogen atom, an alkyl group or a haloalkyl group having 1 to 6 carbon atoms, or an alkoxy group having 1 to 6 carbon atoms),

A molecule of the acacen or a derivative thereof has the smallest structure among many Schiff bases that can bond with oxygen, can coordinate with various metals such as iron, copper, nickel, cobalt, and manganese, and cobalt(II) ions are most preferable in terms of adsorption of oxygen molecules.

The substituents R^{1a}, R^{1b}, R², and R³ in the general formula (1) will be described.

Halogen atoms include fluorine, chlorine, bromine, and iodine.

Examples of the alkyl group having 1 to 6 carbon atoms include straight-chain or branched-chain alkyl groups such as methyl, ethyl, n-propyl, isopropyl, n-butyl, isobutyl, sec-butyl, tert-butyl, n-pentyl, neopentyl, and n-hexyl.

Examples of the haloalkyl group having 1 to 6 carbon atoms include an alkyl group in which any hydrogen atom of the above alkyl group is substituted with the above halogen atom, and specifically include fluoromethyl, chloromethyl, bromomethyl, and trifluoromethyl.

Examples of the alkoxy group having 1 to 6 carbon atoms include straight-chain or branched-chain alkoxy groups such as methoxy, ethoxy, n-propoxy, isopropoxy, n-butoxy, isobutoxy, tert-butoxy, n-pentoxy, and n-hextoxy.

Examples of the acyl group having 2 to 6 carbon atoms include straight-chain or branched-chain aliphatic acyl groups such as acetyl, propionyl, butyryl, isobutyryl, valeryl, isovaleryl, pivaloyl, and caproyl.

Examples of the alkoxycarbonyl group having 2 to 6 carbon atoms include straight-chain or branched-chain alkoxycarbonyl groups such as methoxycarbonyl, ethoxycarbonyl, n-propoxycarbonyl, isopropoxycarbonyl, n-butoxycarbonyl, isobutoxycarbonyl, tert-butyloxycarbonyl, and n-pentoxycarbonyl.

R^{1a} and R^{1b} may be bonded to each other via an atom or an atomic group bonded thereto to form a cycloalkyl ring such as a cyclopentyl ring and a cyclohexyl ring.

The cobalt-acacen complex may be produced, for example, by synthesizing acacen or an acacen derivative through a dehydration condensation reaction with corresponding acetylacetone and ethylenediamine in an alcohol solvent such as ethanol, followed by reacting the obtained acacen or acacen derivative, as a ligand, with cobalt ions under basic conditions. In addition, the cobalt-acacen complex may be produced by simultaneously adding an acetate of cobalt during the synthesis of acacen or an acacen derivative.

On matching with the general formula (1), it is possible to produce the cobalt-acacen complex containing a planar ligand and a cobalt atom by synthesizing, in the solvent as described above, acacen or an acacen derivative through a dehydration condensation reaction between ethylenediamine or an ethylenediamine analogous compound having a substituent represented by general formula (2):

H₂N—CR^{1a}R^{1b}—CR^{1a}R^{1b}—NH₂

(where R^{1a} and R^{1b} are synonymous with those in the general formula (1))
and acetylacetone or an acetylacetone analogous compound having a substituent represented by general formula (3):

R²₃C—CO—CHR³—CO—CR²₃

(where R² and R³ are synonymous with those in the general formula (1)),
followed by reacting the obtained acacen or acacen derivative, as a ligand, with cobalt ions under basic conditions.

The cobalt-acacen complex obtained by such a synthetic method results in various structures by using compounds having substituents in each of acetylacetone and ethylenediamine, which are synthetic raw materials.

Examples of the substitutes for acetylacetone may include methyl acetylacetone, 3-ethyl-2,4-pentandione, 3-chloroacetylacetone, trifluoroacetylacetone, 1,1,5,5-tetrafluoro-2,4-pentandione, hexafluoroacetylacetone, 6-methyl-2,4-heptanedione, 3,5-heptanedione, 2,2-dimethyl-3,5-heptanedione, 1,1,1-trifluoro-2,4-hexanedione, 1,1,1-trifluoro-5-methyl-2,4-hexanedione, 1,1,1-trifluoro-5,5-dimethyl-2,4-hexanedione, 2,6-dimethyl-3,5-heptanedione, 2,2,6,6-tetramethyl-3,5-heptanedione, and 2,2,7-trimethyl-3,5-octanedione.

Examples of the substitute for ethylenediamine may include 1,2-propane diamine, 2-methyl-1,2-propanediamine, 1,2-dimethylethylenediamine, 1,2-cyclohexanediamine, and 1,1,2,2-tetramethylethylenediamine.

Examples of the cobalt acacen derivative obtained by these combinations may include not only the cobalt acacen, that is, N,N′-ethylenebis(acetylacetonylideneaminate)cobalt(II), but also N,N′-ethylenebis(3-methylacetylacetonylideneaminate)cobalt(II), N,N′-ethylenebis(3-chloroacetylacetonylideneaminate)cobalt(II), N,N′-ethylenebis(2,2-dimethylpropionylacetonylideneaminate)cobalt(II), N,N′-ethylenebis(trifluoroacetylacetonylideneaminate)cobalt(II), N,N′-methylethylenebis(acetylacetonylideneaminate)cobalt(II), N,N′-methylethylenebis(3-methylacetylacetonylideneaminate)cobalt(II), N,N′-1,1-dimethylethylenebis(acetylacetonylideneaminate)cobalt(II), and N,N′-1,1,2,2-tetramethylethylenebis(acetylacetonylideneaminate)cobalt(II). In particular, N,N′-ethylenebis(acetylacetonylideneaminate)cobalt(II) is particularly preferable in terms of oxygen absorption.

That is, the cobalt complex comprised of the cobalt atom and the acacen ligand obtained through a dehydration condensation reaction between ethylenediamine and acetylacetone, that is, N,N′-ethylenebis(acetylacetonylideneaminate), is particularly preferable as the cobalt-acacen complex.

### (Ionic Liquid)

An ionic liquid is a substance consisting only of ions, and is often defined as typically having a melting point of 100° C. or lower. The ionic liquid available in the present invention is a substance in which an ionic ligand having an amine structure is paired with a counter ion thereof, either an anion or cation has an amine structure, and the amine structure can be coordinated with a cobalt-acacen complex.

Among ions having the amine structure, ions having a small molecular structure are advantageous in that only a little interference is present due to the three-dimensional structure and accessibility is provided to realize coordination with the cobalt atom. Therefore, it is preferable that the ion is an alkylammonium cation being a general-purpose cation constituting an ionic liquid, and a length of the alkyl chain is 2 to 6 carbon atoms. That is, the ionic ligand is preferably an ammonium cation having an alkyl group having 2 to 6 carbon atoms, and in particular, 1,1,1-trimethylhydrazinium, 1-ethyl-1,1-dimethylhydrazinium, and 1,1-dimethyl-1-pentylhydrazinium cation, in which three methyl groups and one amino group are bonded to nitrogen and the smallest molecular weight is present, are particularly preferable.

Further, among the ions having an amine structure, ions with a high degree of basicity in the amine structure is advantageous in that bondability and adsorption action between the cobalt-acacen complex and the oxygen molecule are enhanced. Therefore, the amine structure of the ionic ligand is preferably a secondary amine.

Similarly, an N-alkyl amino acid having an alkyl chain in the amino group of the amino acid is also suitable for a general-purpose anion constituting an ionic liquid. That is, the ionic ligand is preferably an N-methyl amino acid, and among these, N-methylglycine in which a glycine having a small molecular weight, from among amino acids, and a methyl group are bonded, is particularly preferable.

In addition to the above-mentioned secondary amine, the ionic ligand preferably includes heterocyclic compounds with strong basicity, having a structure such as in imidazole and pyridine. There are various types of ionic functional groups bonded to these heterocyclic compounds, and among these, a carboxylic acid serving as a carbonyl group is easy to be handled and easy to be configured as an ionic liquid with other cation species, similarly to N-methyl amino acids, hence preferable.

Examples of the functional group bonded to the heterocyclic compound include not only a carbonyl group but also halogen atoms such as bromo, chloro, and fluoro, and alkyl groups including methyl, ethyl, and trifluoromethyl. Examples thereof include 1-imidazolecarboxylic acid, 4-imidazolecarboxylic acid, 1-methyl-4-imidazolecarboxylic acid, pyridine-2-carboxylic acid, 4-bromo-2-pyridinecarboxylic acid, 5-bromo-2-pyridinecarboxylic acid, and 6-bromo-2-pyridinecarboxylic acid.

Known cations (counter ions) paired with an ionic ligand anion include imidazole, pyridinium, pyrrolidinium, phosphonium, and ammonium, but in the present invention, from the viewpoint of compatibility, phosphonium and ammonium are preferable, and ammonium or aliphatic quaternary phosphonium having an alkyl chain having 2 to 20 carbon atoms is particularly preferable.

The ionic liquid may be obtained through an anion exchange reaction between the above-mentioned compound serving as an anion and phosphonium salt or ammonium salt serving as a cation.

Examples of the phosphonium salts and ammonium salts include tetramethylphosphonium bromide, tetraethylphosphonium bromide, tetrabutylphosphonium bromide, tetrahexylphosphonium bromide, triethylhexylphosphonium bromide, triethyloctylphosphonium bromide, triethyl(2-methoxyethyl)phosphonium bromide, tributyloctylphosphonium bromide, tributyldodecylphosphonium bromide, tributyl(2-methoxyethyl)phosphonium bromide, trihexyldodecylphosphonium bromide, trihexyl(tetradecyl)phosphonium bromide, and a chloride corresponding to these bromides. In particular, the phosphonium cation is preferably used to be paired with an N-methyl amino acid serving as an ionic ligand.

All combinations of these phosphonium salts or ammonium salts with anions bonded to be axially coordinated with the cobalt complex are not necessarily liquid at ordinary temperature. Therefore, among these, triethylpentylphosphonium bromide, tributyloctylphosphonium bromide, and trihexyl(tetradecyl)phosphonium bromide, having a low melting point and being easy to be an ionic liquid in forming a combination with each anion, are preferable, and in particular, triethylpentylphosphonium bromide, forming an ionic liquid in combination with many anions and having a small molecular weight that is advantageous for low viscosity, is particularly preferable.

On the other hand, well-known anions (counter ions) paired with an ionic ligand cation include tetrafluoroborate, hexafluorophorphosphonate, trifluoroacetic acid, trifluoromethanesulfonate, and bis(trifluoromethanesulfonyl)imide, but in the present invention, bis(trifluoromethanesulfonyl)imide, which has the lowest viscosity, is particularly preferable, and a cation paired with bis(trifluoromethanesulfonyl)imide is preferably an ammonium cation with a secondary amine.

### (Method for Producing Liquid Metal Complex Having Oxygen Absorbing Ability)

For the liquid metal complex having an oxygen absorbing ability according to the present invention, the above-described cobalt-acacen complex and an ionic liquid in which the ionic ligand having an amine structure is paired with the counter ion thereof are mixed in an alcohol solvent such as ethanol and heated and stirred to obtain a coordination structure of a cobalt-acacen complex and an ionic liquid.

A ratio in effective component of the cobalt-acacen complex and the ionic liquid may be a molar equivalent ratio of 1:2, but the effective component of the cobalt-acacen complex may be in excess.

The coordination structure of the cobalt-acacen complex and the ionic liquid may be confirmed by a well-known method such as comprehensive analyses including ultraviolet-visible absorption spectroscopy, ⁵⁹Co-NMR spectroscopy, and changes in color.

The liquid metal complex having an oxygen absorbing ability according to the present invention preferably has a viscosity lower than 10000 mPa s.

If the viscosity of the liquid metal complex exceeds 10000 mPa s, the fluidity of the complex serving as an oxygen carrier and the diffusivity of oxygen may decrease to deteriorate the oxygen absorbing ability.

The viscosity of the liquid metal complex is preferably 100 to 6000 mPa s, more preferably 100 to 4000 mPa s.

The specific viscosities (mPa s) of the liquid metal complex are 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3500, 4000, 4500, 5000, 5500, and 6000.

### EXAMPLES

The present invention will be specifically described below with reference to Examples and Comparative Examples, but the present invention is not limited thereto.

### [Synthesis of Liquid Metal Complex]

### Example 1-1

10.0 g (100 mmol) of acetylacetone [manufactured by Tokyo Chemical Industry Co., Ltd., purity>99%] was added into 50 ml of ethanol, 3.0 g (50 mmol) of ethylenediamine [manufactured by Fuji Film Wako Pure Chemical Corporation, special grade] was added dropwise and mixed, and the mixture was stirred for about 5 hours at room temperature for reaction. A white substance obtained by recrystallization from the reaction solution was washed with diethyl ether to obtain 7.20 g of N,N′-ethylenebis(acetylacetonylideneaminate) (hereinafter referred to as “acacen”).

Next, 2.25 g (10 mmol) of the obtained acacen and 2.49 g (10 mmol) of cobalt(II) acetate tetrahydrate [manufactured by Fuji Film Wako Pure Chemical Corporation, special grade] was added to 20 ml of ethanol and was stirred at 80° C. for 3 hours for reaction to obtain a russet solution containing a cobalt-acacen complex. Next, 4.04 g (20 mmol) of 1,1,1-trimethylhydrazinium iodide [manufactured by Sigma-Aldrich, purity 97%] was added dropwise to the resultant solution and mixed, and further, the mixture was stirred at 80° C. for 3 hours for reaction to obtain a brown solution. Next, the obtained solution was heated and dried at 80° C. for 12 hours to volatilize and remove ethanol serving as a solvent. The remaining brown liquid was vacuum-dried to obtain 3.62 g of a solid serving as a coordination structure of a cobalt-acacen complex and 1,1,1-trimethylhydrazinium iodide.

Next, 3.40 g of the solid of the obtained coordination structure was added with 20 ml of a solution of 2.87 g (10 mmol) of lithium bis(trifluoromethanesulfonyl)imide [manufactured by Tokyo Chemical Industry Co., Ltd., purity>98%], anion exchange reaction was carried out by stirring the mixture at room temperature for 12 hours, and next, the mixture was washed with pure water to obtain 3.12 g of the target liquid (liquid metal complex).

At this time, it was confirmed through ultraviolet-visible absorption spectroscopy, observation of the color development state, and the like that the obtained target liquid had the cations of the ionic liquid coordinated with the cobalt ions of the cobalt-acacen complex.

### Example 1-2

### Synthesis of 1-ethyl-1,1-dimethylhydrazinium bis(trifluoromethanesulfonyl)imide

6.0 g (100 mmol) of 1,1-dimethylhydrazine [manufactured by Tokyo Chemical Industry Co., Ltd., purity>98%] was added with 20 ml of tetrahydrofuran (THF), and the mixture was stirred to prepare a solution. While stirring the THF solution, 14.9 g (20 mmol) of 1-bromoethane [manufactured by Tokyo Chemical Industry Co., Ltd., purity>98%] was added dropwise. After completion of the dropping, the mixture was stirred at room temperature for 6 hours. After completion of the reaction, the obtained white solid was filtered and washed by applying 10 ml of THE onto the filter paper. Subsequently, any remaining air is expelled through vacuum dehydration for 12 hours to remove the solvent to obtain 1-ethyl-1,1-dimethylhydrazinium bromide. Identification was performed using NMR.

3.1 g of the obtained 1-ethyl-1,1-dimethylhydrazinium bromide was dissolved in pure water, 5.1 g (18 mmol) of lithium bis(trifluoromethanesulfonyl)imide was added to this aqueous solution, and the mixture was stirred at room temperature for 3 hours. The precipitated white solid was filtered and washed by applying pure water onto the filter paper. Next, as a result of being deaerated and dried at 80° C. for 12 hours, 5.92 g of the target 1-ethyl-1,1-dimethylhydrazinium bis(trifluoromethanesulfonyl)imide was obtained as a white solid.

1.35 g (6 mmol) of acacen obtained by synthesis in much the same manner as in Example 1-1 and 1.50 g (6 mmol) of cobalt(II) acetate tetrahydrate were added to 20 ml of ethanol and stirred at 80° C. for 3 hours for reaction to obtain a russet solution containing a cobalt-acacen complex. The obtained solution was added with 4.43 g (12 mmol) of 1-ethyl-1,1-dimethyihydrazinium bis(trifluoromethanesulfonyl)imide, and the mixture was stirred at 80° C. for 3 hours to obtain a brown solution. The obtained solution was heated and dried at 80° C. for 12 hours to volatilize and remove ethanol serving as a solvent to obtain 5.82 g of the target liquid (liquid metal complex).

In much the same manner as in Example 1-1, it was confirmed that the obtained target liquid had a constituent cation coordinated with the cobalt ion of the cobalt-acacen complex.

### Example 1-3

### Synthesis of 1,1-dimethyl-1-pentylhydrazinium bis(trifluoromethanesulfonyl)imide

1.2 g (20 mmol) of 1,1-dimethylhydrazine [manufactured by Tokyo Chemical Industry Co., Ltd., purity>98%] was added with 5 ml of tetrahydrofuran (THF), and the mixture was stirred to prepare a solution. While stirring the THF solution, 3.1 g (20 mmol) of 1-bromopentane [manufactured by Tokyo Chemical Industry Co., Ltd., purity>98%] was added dropwise. After the addition was all completed, the solution was heated to 50° C. and stirred for 12 hours. After completion of the reaction, separation into two layers was observed in the solution. After the solution was naturally cooled to room temperature, the target liquid was washed with diethyl ether. The solution was dried by being heated at 60° C. to obtain 1,1-dimethyl-1-pentylhydrazinium bromide. Identification was performed using NMR.

3.5 g of the obtained 1,1-dimethyl-1-pentylhydrazinium bromide was dissolved in pure water to prepare an aqueous solution. It was confirmed that when 5.1 g (18 mmol) of lithium bis(trifluoromethanesulfonyl)imide was added to the aqueous solution, oil droplets were immediately precipitated. The oil droplets were extracted with dichloromethane and washed three times with pure water. Dichloromethane was removed by an evaporator to obtain 6.59 g of the target ionic liquid of 1,1-dimethyl-1-pentylhydrazinium bis(trifluoromethanesulfonyl)imide.

1.35 g (6 mmol) of acacen obtained by synthesis in much the same manner as in Example 1-1 and 1.50 g (6 mmol) of cobalt(II) acetate tetrahydrate were added to 20 ml of ethanol and stirred at 80° C. for 3 hours for reaction to obtain a russet solution containing a cobalt-acacen complex. The obtained solution was added with 4.93 g (12 mmol) of 1,1-dimethyl-1-pentylhydrazinium bis(trifluoromethanesulfonyl)imide, and the mixture was stirred at 80° C. for 3 hours to obtain a brown solution. The obtained solution was heated and dried at 80° C. for 12 hours to volatilize and remove ethanol serving as a solvent to obtain 6.23 g of the target liquid (liquid metal complex).

In much the same manner as in Example 1-1, it was confirmed that the obtained target liquid had a constituent cation coordinated with the cobalt ion of the cobalt-acacen complex.

### Example 2

### Synthesis of triethylpentylphosphonium bromide

100 ml of 1.0 M THE solution of triethylphosphine [manufactured by Sigma-Aldrich, purity 97%] was placed in a three-neck flask and heated to 70° C. to obtain a reflux state. Under reflux, 15.58 g (103 mmol) of 1-bromopentane [manufactured by Tokyo Chemical Industry Co., Ltd., purity>98%] was added dropwise, and then, stirred at 80° C. for 6 hours for reaction. After completion of the reaction, it was observed that a white solid was produced.

The obtained reaction solution was cooled to room temperature, and subsequently, was added dropwise to about 300 ml of hexane with stirring, and then stirred for 1 hour to sufficiently precipitate a solid. The suspension was left statically overnight to allow the solid to settle. The resulting solid product was transferred to an eggplant flask, and hexanes were removed by depressurizing at 40° C. for about 3 hours by using an evaporator to obtain 17.42 g of triethylpentylphosphonium bromide as a white solid.

100 ml of ethanol was added and stirred with 2.70 g (10 mmol) of the obtained triethylpentylphosphonium bromide and 20 g of anion exchange resin [Amberlite (registered trademark) TRN78 Hydroxide Form, manufactured by Sigma-Aldrich] to carry out a substitution reaction on the hydroxide. Next, the obtained reaction solution was filtered and separated by suction filtration, the obtained filtrate was added, for reaction, with an aqueous solution prepared by dissolving 0.98 g (11 mmol) of N-methylglycine [manufactured by Tokyo Chemical Industry Co., Ltd., purity>98%] into 20 ml of pure water, and the solvent and unreacted substances were removed by concentration under reduced pressure to obtain 2.64 g of an ionic liquid containing a triethylpentylphosphonium cation and an N-methylglycine anion.

In much the same manner as in Example 1-1, a solution of cobalt-acacen complex prepared by charging 50 ml of ethanol with the same molar amount of acacen and cobalt(II) acetate tetrahydrate was added with 2.60 g of the prepared ionic liquid, and stirred at 80° C. for 3 hours for reaction to obtain 5.28 g of the target liquid (liquid metal complex).

### Example 3

In much the same way as in Example 2, 3.95 g (10 mmol) of tributyl-n-octylphosphonium bromide [Manufactured by Tokyo Chemical Industry Co., Ltd., purity>98%] and 0.98 g (11 mmol) of N-methylglycine [manufactured by Tokyo Chemical

Industry Co., Ltd., purity>98%] were reacted to obtain 4.12 g of ionic liquid. In much the same manner as in Example 1-1, a solution of cobalt-acacen complex prepared by charging 50 ml of ethanol with the same molar amount of acacen and cobalt(II) acetate tetrahydrate was added with 4.08 g of the prepared ionic liquid, and stirred at 80° C. for 3 hours for reaction to obtain 7.48 g of the target liquid (liquid metal complex).

### Example 4-1

In much the same way as in Example 2, 5.64 g (10 mmol) of trihexyl(tetradecyl)phosphonium bromide [manufactured by Sigma-Aldrich, purity>95%] and 0.98 g (11 mmol) of N-methylglycine [manufactured by Tokyo Chemical Industry Co., Ltd., purity>98%] were reacted to obtain 5.31 g of ionic liquid.

In much the same manner as in Example 1-1, 5.28 g of the prepared solution of cobalt-acacen complex was added with the prepared ionic liquid and stirred at 80° C. for 3 hours for reaction to obtain 9.64 g of the target liquid (liquid metal complex).

### (Example 4-2) [P₆₆₆₁₄]₂[Co(acacen)(N-mGly)(Tf₂N)]

### Synthesis of trihexyl(tetradecyl)phosphonium N-methylglycinate

In much the same manner as in Example 4-1, 4.62 g of an ionic liquid containing a trihexyl(tetradecyl)phosphonium cation and an N-methylglycine anion was obtained.

### Synthesis of trihexyl(tetradecyl)phosphonium bis(trifluoromethanesulfonyl)imide

5.64 g (10 mmol) of trihexyl(tetradecyl)phosphonium bromide was added to 50 ml of pure water and stirred to form an emulsion, followed by adding thereto 3.45 g (12 mmol) of lithium bis(trifluoromethanesulfonyl)imide and stirring the mixture at room temperature for 2 hours for reaction.

After the reaction is completed, by adding dichloromethane, the target reaction product was extracted to the dichloromethane phase. Subsequently, the dichloromethane phase was separated from the aqueous phase, and then, washed with pure water.

The resulting dichloromethane solution was transferred to an eggplant flask, and depressurized at 40° C. for about 3 hours using an evaporator to remove dichloromethane, any remaining air is expelled through vacuum dehydration for 12 hours, and as a result, 6.85 g of trihexyl(tetradecyl)phosphonium bis(trifluoromethanesulfonyl)imide was obtained as a viscous liquid.

1.35 g (6 mmol) of acacen obtained by synthesis in much the same manner as in Example 1-1 and 1.50 g (6 mmol) of cobalt(II) acetate tetrahydrate were added to 20 ml of ethanol and stirred at 80° C. for 3 hours for reaction to obtain a russet solution containing a cobalt-acacen complex. The obtained solution was added with 3.43 g (6 mmol) of trihexyl(tetradecyl)phosphonium N-methylglycinate and stirred at 80° C. for 3 hours, and thereafter, 4.59 g (6 mmol) of trihexyl(tetradecyl)phosphonium bis(trifluoromethanesulfonyl)imide was added to the reaction solution, and the mixture was further stirred for 2 hours. The obtained solution was heated and dried at 80° C. for 12 hours to volatilize and remove ethanol serving as a solvent to obtain 8.12 g of the target liquid (liquid metal complex).

In much the same manner as in Example 1-1, it was confirmed that the obtained target liquid had a constituent cation coordinated with the cobalt ion of the cobalt-acacen complex.

### Comparative Example 1

The liquid metal complex was obtained in much the same manner as in Example 2 except that a cobalt-salen complex was used instead of the cobalt-acacen complex.

Specifically, in much the same manner as in Example 2, after preparing an ionic liquid containing a triethylpentylphosphonium cation and an N-methylglycine anion, 5.42 g of the obtained ionic liquid and 3.25 g (10 mmol) of N,N′-bis(salicylidene)ethylenediaminocobalt(II) [manufactured by Tokyo Chemical Industry Co., Ltd., purity>95%], which were added in 100 ml of ethanol, were stirred and mixed for 3 hours at room temperature for reaction, and the solvent and unreacted substances were removed by concentration under reduced pressure to obtain 3.86 g of the target liquid (liquid metal complex).

### Comparative Example 2

The liquid metal complex was obtained in much the same manner as in Example 3 except that a cobalt-salen complex was used instead of the cobalt-acacen complex.

Specifically, in much the same manner as in Example 3, after preparing an ionic liquid containing a tributyloctylphosphonium cation and an N-methylglycine anion, 8.66 g of the obtained ionic liquid and 3.25 g (10 mmol) of N,N′-bis(salicylidene)ethylenediaminocobalt(II) [manufactured by Tokyo Chemical Industry Co., Ltd., purity>95%], which were added in 100 ml of ethanol, were stirred and mixed for 3 hours at room temperature for reaction, and the solvent and unreacted substances were removed by concentration under reduced pressure to obtain 6.14 g of the target liquid (liquid metal complex).

### Comparative Example 3

The liquid metal complex was obtained in much the same manner as in Example 4-1 except that a cobalt-salen complex was used instead of the cobalt-acacen complex.

Specifically, in much the same manner as in Example 4-1, after preparing an ionic liquid containing a trihexyl(tetradecyl)phosphonium cation and an N-methylglycine anion, 11.8 g of the obtained ionic liquid and 3.25 g (10 mmol) of N,N′-bis(salicylidene)ethylenediaminocobalt(II) [manufactured by Tokyo Chemical Industry Co., Ltd., purity>95%], which were added in 100 ml of ethanol, were stirred and mixed for 3 hours at room temperature for reaction, and the solvent and unreacted substances were removed by concentration under reduced pressure to obtain 7.88 g of the target liquid (liquid metal complex).

### [Evaluation of Viscosity]

The viscosities of the liquid metal complexes of the cobalt-acacen complexes obtained in all the Examples and the liquid metal complexes of the cobalt-salen complexes obtained in Comparative Examples 1 to 3 were measured under the condition of a temperature of 30° C. by using an EMS viscometer (manufactured by Kyoto Electronics Manufacturing Co., Ltd., model: EMS-1000).

The obtained results are shown in Table 1 together with the structural formula of the liquid metal complex.

### [Evaluation of Oxygen Absorption Amount]

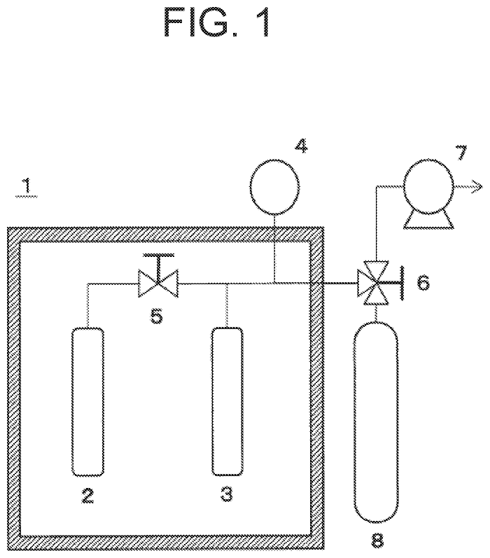

The oxygen absorption amounts of the liquid metal complexes of the cobalt-acacen complexes obtained in all the Examples and the liquid metal complexes of the cobalt-salen complexes obtained in Comparative Examples 1 to 3 were measured by using an absorption test device illustrated in FIG. 1.

FIG. 1 is a schematic diagram of the absorption test device for measuring an oxygen absorption amount, and illustrates an absorption test device (constant temperature chamber) 1, a sample cell 2, a control cell 3, a pressure gauge 4, a two-way valve 5, a three-way valve 6, a vacuum pump 7, and an oxygen or nitrogen gas supply source (cylinder) 8 with corresponding reference numerals.

Air in the absorption test device was replaced with nitrogen, and 3.02 g of a sample liquid was introduced into the device using a syringe. Subsequently, nitrogen substitution was further carried out, and the inside of the device was deaerated for 1 hour or more to dry the inside of the system. Subsequently, oxygen gas was introduced at a predetermined pressure of 0 to 20 kPa under the condition of a temperature of 30° C., a pressure change due to absorption was measured by a pressure sensor, and the oxygen absorption amount was estimated from the results.

The obtained results are shown in Table 1 together with the structural formula of the liquid metal complex.

TABLE 1

| | | | Oxygen |
|---|---|---|---|
| | | Viscosity | absorption amount |
| | Structural formula | (mPa · s) | mol-O₂/mol-Co(II) |
| Example 1-1 | [Co(acacen)(aN₁₁₁)₂][Tf₂N]₂ | 4.0 × 10² | 0.02 |
| Example 1-2 | [Co(acacen)(aN₁₁₂)₂][Tf₂N]₂ | 3.2 × 10² | 0.02 |
| Example 1-3 | [Co(acacen)(aN₁₁₅)₂][Tf₂N]₂ | 2.3 × 10² | 0.03 |
| Example 2 | [P₂₂₂₅]₂[Co(acacen)(N-mGly)₂] | 2.3 × 10³ | 0.30 |
| Example 3 | [P₄₄₄₈]₂[Co(acacen)(N-mGly)₂] | 3.8 × 10³ | 0.32 |
| Example 4-1 | [P₆₆₆₁₄]₂[Co(acacen)(N-mGly)₂] | 5.2 × 10³ | 0.33 |
| Example 4-2 | [P₆₆₆₁₄]₂[Co(acacen)(N-mGly)(Tf₂N)] | 1.4 × 10³ | 0.33 |
| Comparative | [P₂₂₂₅]₂[Co(salen)(N-mGly)₂] | 1.6 × 10⁴ | 1.11 |
| Example 1 | | | |
| Comparative | [P₄₄₄₈]₂[Co(salen)(N-mGly)₂] | 2.6 × 10⁴ | 0.87 |
| Example 2 | | | |
| Comparative | [P₆₆₆₁₄]₂[Co(salen)(N-mGly)₂] | 4.6 × 10⁴ | 0.67 |
| example 3 | | | |

The abbreviations of the structural formulas in Table 1 are as follows.
Acacen: N,N′-ethylenebis(acetylacetonylideneaminate)
Salen: N, N′-bis(sari chi dene)ethylenediamine
aN₁₁₁: 1,1,1-trimethylhydrazinium cation
aN₁₁₂: 1-ethyl-1,1-dimethylhydrazinium cation
aN₁₁₅: 1,1-dimethyl-1-pentylhydrazinium cation
P₂₂₂₅: triethylpentylphosphonium cation
P₄₄₄₈: tributyloctylphosphonium cation
P₆₆₆₁₄: trihexyl(tetradecyl)phosphonium cation
Tf₂N: bis(trifluoromethanesulfonyl)imide
N-mGly: N-methylglycinate

The following can be seen from the results in Table 1.
The liquid metal complexes according to the present invention (all Examples) are all in a viscosity region exhibiting an appropriate fluidity of 10000 mPa-s or less.
The liquid metal complexes according to the present invention (Examples 2, 3, and 4-1) each have a significantly lower viscosity, compared with the liquid metal complexes (Comparative Examples 1 to 3) in which the same ionic ligands are combined with conventional metal complexes.
The liquid metal complexes according to the present invention (Examples 1-1, 1-2, and 1-3) have a low viscosity of 1000 mPa s or less, which indicates considerably high fluidity as a liquid containing a complex structure.

The liquid metal complexes according to the present invention (all Examples) each have an oxygen absorbing ability, but the oxygen absorption amount is not necessarily large. However, the membrane performance obtained when each of the liquid metal complexes is applied to an oxygen separation membrane depends on the oxygen absorption performance and permeation/diffusion performance. Therefore, if the improvement of oxygen permeation/diffusion performance as a result of a decreased viscosity of the complex serving as an oxygen carrier contributes more greatly than oxygen absorption performance, the membrane performance will be improved.

The liquid metal complexes of all Examples are examples of low-viscosity liquid complex structures in which the permeation and diffusion of oxygen via oxygen carriers is fully functional, and when the amine structure of the ionic ligand is modified, it is possible to obtain a liquid metal complex having higher oxygen absorption in addition to low viscosity.

### INDUSTRIAL APPLICABILITY

The liquid metal complex having an oxygen absorbing ability according to the present invention can be applied to fields requiring an oxygen absorbing material and an oxygen separation membrane, for the purpose of oxygen separation, concentration, removal, storage, or the like.

### DESCRIPTION OF REFERENCE NUMERALS

1 Absorption test device (constant temperature chamber)
2 Sample cell
3 Control cell
4 Pressure gauge
5 Two-way valve
6 Three-way valve
7 Vacuum pump
8 Oxygen or nitrogen gas supply source (cylinder)

1. A liquid metal complex having an oxygen absorbing ability, the liquid metal complex containing a cobalt-acacen complex or a derivative thereof, and an ionic liquid in which an ionic ligand having an amine structure and a counter ion thereof are paired, wherein
the cobalt-acacen complex or the derivative thereof is expressed by general formula (1): 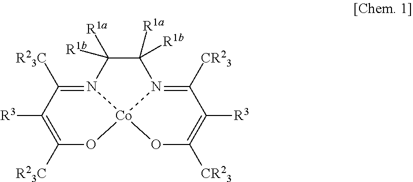
(where R^{1a}, R^{1b} and R³ are each independently a hydrogen atom, a halogen atom, an alkyl group or a haloalkyl group having 1 to 6 carbon atoms, an alkoxy group having 1 to 6 carbon atoms, an acyl group having 2 to 6 carbon atoms, or an alkoxycarbonyl group having 2 to 6 carbon atoms, R^{1a} and R^{1b} may be bonded to each other via an atom or an atomic group bonded to R^{1a} and R^{1b} to form a cycloalkyl ring, and R² is independently a hydrogen atom, a halogen atom, an alkyl group or a haloalkyl group having 1 to 6 carbon atoms, or an alkoxy group having 1 to 6 carbon atoms),
and
the liquid metal complex has a structure in which the amine structure of the ionic ligand is axially coordinated with a cobalt atom of the cobalt-acacen complex or the derivative thereof.

2. The liquid metal complex having an oxygen absorbing ability according to claim 1, wherein the ionic ligand is an ammonium cation having an alkyl group having 2 to 6 carbon atoms.

3. The liquid metal complex having an oxygen absorbing ability according to claim 1, wherein the amine structure of the ionic ligand is a secondary amine.

4. The liquid metal complex having an oxygen absorbing ability according to claim 1, wherein the ionic ligand is an N-methyl amino acid.

5. The liquid metal complex having an oxygen absorbing ability according to claim 1, wherein the ionic ligand is a heterocyclic compound containing a structure of imidazole or pyridine.

6. The liquid metal complex having an oxygen absorbing ability according to claim 1, wherein the counter ion contains an anion of bis(trifluoromethanesulfonyl)imide.

7. The liquid metal complex having an oxygen absorbing ability according to claim 1, wherein the counter ion contains a phosphonium cation.

8. The liquid metal complex having an oxygen absorbing ability according to claim 1, wherein the cobalt-acacen complex is a cobalt complex containing a cobalt atom and an acacen ligand obtained through a dehydration condensation reaction of ethylenediamine and acetylacetone.

9. The liquid metal complex having an oxygen absorbing ability according to claim 1, wherein the liquid metal complex having an oxygen absorbing ability has a viscosity lower than 10000 mPa·s.
